(19) United States
(12) Patent Application Publication
(10) Pub. No.: US 2021/0062059 A1
(43) Pub. Date: Mar. 4, 2021

(54) COOLANT COMPOSITION AND COOLING SYSTEM

(71) Applicant: TOYOTA JIDOSHA KABUSHIKI KAISHA, Toyota-shi (JP)

(72) Inventors: Yasuaki KODAMA, Seto-shi (JP); Masao WATANABE, Okazaki-shi (JP); Tomoharu KATAOKA, Shizuoka-shi (JP); Daisuke TOKOZAKURA, Susono-shi (JP)

(21) Appl. No.: 16/939,222

(22) Filed: Jul. 27, 2020

(30) Foreign Application Priority Data
Aug. 26, 2019 (JP) 2019-153743

(51) Int. Cl. C09K 5/10; C07C 69/14; C07C 69/017

(52) U.S. Cl. CPC C09K 5/10; B60K 11/04; C07C 69/017; C07C 69/14

(57) ABSTRACT

This disclosure provides a nonaqueous coolant composition excellent in insulation property and heat transfer characteristic. The embodiment is a coolant composition that includes at least one carboxylic acid ester compound as a nonaqueous base and is substantially free of water.

### CROSS REFERENCE TO RELATED APPLICATIONS

The present application claims priority from Japanese patent application JP 2019-153743 filed on Aug. 26, 2019, the entire content of which is hereby incorporated by reference into this application.

### BACKGROUND

### Technical Field

The present disclosure relates to a coolant composition and a cooling system that use the coolant composition.

### Background Art

A vehicle with traction motor, such as a hybrid vehicle and an electric vehicle, includes a power control unit (PCU) for appropriately controlling an electric power. The PCU includes an inverter that drives the motor, a boost converter that controls a voltage, a DC/DC converter that steps down a high voltage, and the like. The inverter or the converter includes a power card as a card-type power module that includes semiconductor devices, and the power card generates a heat caused by its switching action. Therefore, the inverter and the converter are equipment that possibly becomes to have a high temperature due to the heat generation. Heat generation equipment in the vehicle with traction motor includes a battery in addition to the inverter and the converter. Accordingly, the vehicle with traction motor includes a cooling system for cooling the inverter, the converter, the battery, and the like.

For example, JP 2017-017228 A discloses a configuration of a semiconductor apparatus used for an inverter of a drive system in a vehicle with traction motor (for example, an electric vehicle or a hybrid vehicle) (FIG. 1). A semiconductor apparatus 2 of FIG. 1 is a unit where a plurality of power cards 10 and a plurality of coolers 3 are stacked. In FIG. 1, reference numeral 10 is attached to only one power card, and reference numerals to the other power cards are omitted. For showing the whole semiconductor apparatus 2, a case 31, which houses the semiconductor apparatus 2, is illustrated by dotted lines. The one power card 10 is sandwiched between the two coolers 3. An insulating plate 6a is sandwiched between the power card 10 and one of the coolers 3, and an insulating plate 6b is sandwiched between the power card 10 and the other of the coolers 3. Greases are applied between the power card 10 and the insulating plates 6a and 6b. Greases are applied also between the insulating plates 6a and 6b and the coolers 3. For easy understanding, FIG. 1 illustrates the one power card 10 and the insulating plates 6a and 6b extracted from the semiconductor apparatus 2. The power card 10 houses a semiconductor device. The power card 10 is cooled by a refrigerant passing through the cooler 3. The refrigerant is a liquid, typically water. The power cards 10 and the coolers 3 are alternately stacked, and the coolers 3 are positioned at both ends in a stacking direction of the unit. The plurality of coolers 3 are coupled by coupling pipes 5a and 5b. A refrigerant supply pipe 4a and a refrigerant discharge pipe 4b are coupled to the cooler 3 positioned at the one end in the stacking direction of the unit. The refrigerant supplied through the refrigerant supply pipe 4a is distributed to every cooler 3 through the coupling pipes 5a. The refrigerant absorbs the heat from the adjacent power card 10 while passing through each cooler 3. The refrigerant that has passed through each cooler 3 passes through the coupling pipe 5b and is discharged from the refrigerant discharge pipe 4b.

Meanwhile, JP 2005-203148 A discloses a coolant that includes a nonaqueous base, and the nonaqueous base specifically includes alkyl benzene, dimethyl silicone, and perfluorocarbon.

### SUMMARY

As the configuration of the semiconductor apparatus disclosed in JP 2017-017228 A, generally, the refrigerant circulates near the power cards or the batteries. Therefore, in the vehicle with traction motor, such as the hybrid vehicle and the electric vehicle, when the coolant leaks due to an accident, the leaked refrigerant possibly contacts a terminal of the power card, the battery, or the like to cause a short circuit. Therefore, from an aspect to reduce the occurrence of the secondary accident in the case of the refrigerant leakage, the refrigerant is desired to have an excellent insulation property. JP 2005-203148 A uses a silicone oil, such as dimethyl silicone, and the silicone oil is excellent from the aspect of the insulation property. However, the silicone oil is significantly low in cooling performance compared with an aqueous refrigerant.

Therefore, the present disclosure provides a nonaqueous coolant composition excellent in insulation property and heat transfer characteristic.

Exemplary aspects of the embodiment will be described as follows.

(1) A coolant composition that comprises at least one carboxylic acid ester compound as a nonaqueous base, wherein the coolant composition is substantially free of water.
(2) The coolant composition according to (1) wherein the carboxylic acid ester compound comprises at least one selected from the group consisting of an aliphatic carboxylic acid ester compound and an aromatic carboxylic acid ester compound, the aliphatic carboxylic acid ester compound is an ester of a saturated or unsaturated aliphatic carboxylic acid having 2 to 30 carbon atoms and a saturated or unsaturated fatty alcohol having 1 to 30 carbon atoms, and the aromatic carboxylic acid ester compound is an ester of an aromatic carboxylic acid having 6 to 20 carbon atoms and a saturated or unsaturated fatty alcohol having 1 to 30 carbon atoms.
(3) The coolant composition according to (1) wherein the carboxylic acid ester compound comprises at least one selected from an aliphatic carboxylic acid ester compound, the aliphatic carboxylic acid ester compound is an ester of a saturated or unsaturated aliphatic carboxylic acid having 2 to 30 carbon atoms and a saturated or unsaturated fatty alcohol having 1 to 30 carbon atoms.
(4) The coolant composition according to (1) wherein the carboxylic acid ester compound comprises at least one selected from an aromatic carboxylic acid ester compound, the aromatic carboxylic acid ester compound is an ester of an aromatic carboxylic acid having 6 to 20 carbon atoms and a saturated or unsaturated fatty alcohol having 1 to 30 carbon atoms.
(5) The coolant composition according to any one of (1) to (4) wherein the saturated or unsaturated fatty alcohol comprises a monohydric alcohol.
(6) The coolant composition according to any one of (1) to (5) wherein a content of the carboxylic acid ester compound in the coolant composition is 10 mass % or more.
(7) The coolant composition according to any one of (1) to (6) that further comprises a mineral oil.
(8) The coolant composition according to (7) wherein a content of the carboxylic acid ester compound in the coolant composition is 10 to 90 mass %, and a content of the mineral oil in the coolant composition is 10 to 90 mass %.
(9) The coolant composition according to (7) wherein a content of the carboxylic acid ester compound in the coolant composition is 30 to 70 mass %, and a content of the mineral oil in the coolant composition is 30 to 70 mass %.
(10) The coolant composition according to any one of (1) to (9) wherein a conductivity at 20° C. is 0.1 μS/cm or less.
(11) The coolant composition according to any one of (1) to (10) wherein a conductivity at 20° C. is 0.001 μS/cm or less.
(12) A cooling system that uses the coolant composition according to any one of (1) to (11) as a refrigerant.
(13) The cooling system according to (12) for cooling heat generation equipment mounted to a vehicle with traction motor.
(14) The cooling system according to (13) wherein the heat generation equipment is an inverter, a converter, a generator, a motor, or a battery.
(15) The cooling system according to (13) or (14) wherein the heat generation equipment includes a power card, and the coolant composition is in physical contact with the power card.

The present disclosure can provide the nonaqueous coolant composition excellent in insulation property and heat transfer characteristic.

### BRIEF DESCRIPTION OF THE DRAWINGS

FIG. 1 is a schematic perspective view illustrating an exemplary configuration of a semiconductor apparatus used for an inverter of a drive system in a vehicle with traction motor.

### DETAILED DESCRIPTION

### 1. Coolant Composition

The embodiment is a coolant composition that comprises at least one carboxylic acid ester compound as a nonaqueous base and is substantially free of water.

The coolant composition according to the embodiment is excellent in insulation property and heat transfer characteristic. Especially, since the coolant composition according to the embodiment is extremely excellent in insulation property, a secondary accident, such as a short circuit, can be suppressed when the coolant composition leaks due to an accident or the like. Therefore, the coolant composition according to the embodiment is usable in a vehicle with traction motor, such as a hybrid vehicle and an electric vehicle in some embodiments.

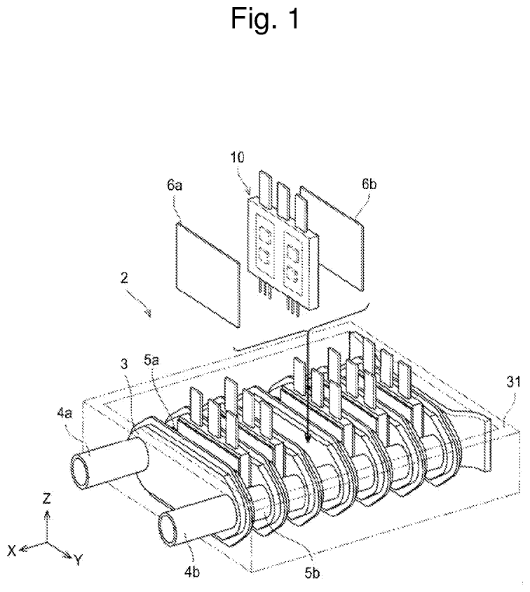

The coolant composition according to the embodiment provides another effect as follows. Conventionally, a typically used ethylene glycol based aqueous coolant has an excellent heat transfer characteristic but has a poor insulation property. Therefore, as illustrated in FIG. 1, a component side of a cooling object needed to have an insulation structure. Specifically, as illustrated in FIG. 1, it was necessary to dispose the insulating plates (6a and 6b of FIG. 1) to ensure the insulation between the electronic equipment and the coolant composition. However, disposing the insulating plates degrades the heat transfer characteristic between the coolant composition and the electronic equipment, thus consequently reducing the cooling performance. Since the coolant composition according to the embodiment is excellent in insulation property, the disposing of the insulating plates can be eliminated, and as a result, a cooling system excellent in cooling performance can be provided.

The coolant composition according to the embodiment provides another effect as follows. As an exemplary means for cooling the electronic equipment, there has been a method to at least partially (partially or completely) immerse the electronic equipment in the coolant composition. For example, for the cooling, the power card can be disposed to be in physical contact with the coolant composition. While this cooling structure has an extremely excellent heat transfer efficiency, the coolant composition requires the extremely excellent insulation property because the electronic equipment and coolant composition are in direct contact. The coolant composition according to the embodiment is extremely excellent in insulation property, non-toxic, and less likely to cause corrosion. Thus, the coolant composition according to the embodiment is usable in the cooling system that has this cooling structure in some embodiments.

The coolant composition according to the embodiment includes the nonaqueous base as the component and is substantially free of water.

In this description, “substantially free of water” means that the coolant composition does not include water in a content range in which expression of the effect of the embodiment is interfered, may mean that the water content in the coolant composition is 1.0 mass % or less, may mean that the water content in the coolant composition is 0.5 mass % or less, may mean that the water content in the coolant composition is 0.1 mass % or less, or may mean that the water content in the coolant composition is 0 mass % (undetectable).

The coolant composition according to the embodiment comprises at least one carboxylic acid ester compound as the nonaqueous base. The carboxylic acid ester compound is excellent in insulation property and heat transfer characteristic. One carboxylic acid ester compound may be used alone, or two or more carboxylic acid ester compounds may be used in combination.

The carboxylic acid ester compound may comprise at least one selected from the group consisting of an aliphatic carboxylic acid ester compound and an aromatic carboxylic acid ester compound. The aliphatic carboxylic acid ester compound is an ester of a saturated or unsaturated aliphatic carboxylic acid having 2 to 30 carbon atoms and a saturated or unsaturated fatty alcohol having 1 to 30 carbon atoms. The aromatic carboxylic acid ester compound is an ester of an aromatic carboxylic acid having 6 to 20 carbon atoms and a saturated or unsaturated fatty alcohol having 1 to 30 carbon atoms.

As described above, the aliphatic carboxylic acid ester compound is the ester of the saturated or unsaturated aliphatic carboxylic acid having 2 to 30 carbon atoms and the saturated or unsaturated fatty alcohol having 1 to 30 carbon atoms. The saturated or unsaturated aliphatic carboxylic acid has the carbon atoms of 2 or more, and the number of carbon atoms may be 4 or more, 6 or more, 7 or more, or 8 or more. The saturated or unsaturated aliphatic carboxylic acid has the carbon atoms of 30 or less, and the number of carbon atoms may be 26 or less, 22 or less, 18 or less, 14 or less, or 10 or less. The saturated or unsaturated fatty alcohol has the carbon atoms of 1 or more, and the number of carbon atoms may be 2 or more. The saturated or unsaturated fatty alcohol has the carbon atoms of 30 or less, and the number of carbon atoms may be 26 or less, 22 or less, 18 or less, 14 or less, 10 or less, or 6 or less. One aliphatic carboxylic acid ester compound may be used alone, or two or more aliphatic carboxylic acid ester compounds may be used in combination.

The aliphatic carboxylic acid is a carboxylic acid in which a carboxyl group is bonded to a hydrocarbon. The aliphatic carboxylic acid includes, for example, an aliphatic monocarboxylic acid, an aliphatic dicarboxylic acid, or a mixture of them. The aliphatic carboxylic acid is the aliphatic monocarboxylic acid in some embodiments. The fatty alcohol includes, for example, an aliphatic monohydric alcohol, an aliphatic dihydric alcohol, or a mixture of them. The fatty alcohol is the aliphatic monohydric alcohol in some embodiments.

The saturated aliphatic carboxylic acid includes, for example, acetic acid, propionic acid, butyric acid, isobutyric acid, pivalic acid, valeric acid, hexanoic acid, heptanoic acid, octanoic acid (caprylic acid), 2-ethylhexanoic acid, decanoic acid, undecanoic acid, dodecanoic acid (lauric acid), hexadecane acid (palmitic acid), octadecanoic acid (stearic acid), malonic acid, succinic acid, glutaric acid, adipic acid, pimelic acid, suberic acid, azelaic acid, sebacic acid, or a mixture of them. The unsaturated aliphatic carboxylic acid includes, for example, oleic acid, undecene acid, erucic acid, linoleic acid, linolenic acid, eleostearic acid, fumaric acid, or a mixture of them. One of them may be used alone, or two or more may be used in combination.

The saturated fatty alcohol includes, for example, methyl alcohol, ethyl alcohol, butyl alcohol, isopropyl alcohol, 2-ethyl hexyl alcohol, octyl alcohol, lauryl alcohol, palmityl alcohol, stearyl alcohol, or a mixture of them. The unsaturated fatty alcohol includes, for example, oleyl alcohol. One of them may be used alone, or two or more may be used in combination.

The aliphatic carboxylic acid ester compound includes an aliphatic monocarboxylic acid ester compound or an aliphatic dicarboxylic acid ester compound. The aliphatic carboxylic acid ester compound is, for example, methyl butyrate, ethyl butyrate, butyl butyrate, methyl valerate, ethyl valerate, methyl hexanoate, ethyl hexanoate, propyl hexanoate, butyl hexanoate, methyl 2-ethylhexanoate, methyl heptanoate, ethyl heptanoate, methyl octanoate, ethyl octanoate, methyl decanoate, ethyl decanoate, methyl undecanoate, ethyl undecanoate, methyl stearate, ethyl stearate, dimethyl malonate, diethyl malonate, dimethyl succinate, diethyl succinate, dimethyl glutarate, diethyl glutarate, dimethyl adipate, diethyl adipate, dimethyl pimelate, diethyl pimelate, dimethyl suberate, diethyl suberate, dimethyl azelate, diethyl azelate, dimethyl sebacate, diethyl sebacate, or a mixture of them. One of them may be used alone, or two or more may be used in combination.

As described above, the aromatic carboxylic acid ester compound is the ester of the aromatic carboxylic acid having 6 to 20 carbon atoms and the saturated or unsaturated fatty alcohol having 1 to 30 carbon atoms. The aromatic carboxylic acid has six or more carbon atoms, and the number of carbon atoms may be seven or more. The aromatic carboxylic acid has 20 or less carbon atoms, and the number of carbon atoms may be 18 or less, 16 or less, 14 or less, 12 or less, or 10 or less. The saturated or unsaturated fatty alcohol can include those described for the aliphatic carboxylic acid ester compound.

The aromatic carboxylic acid is, for example, an aromatic monocarboxylic acid or an aromatic dicarboxylic acid, and is the aromatic monocarboxylic acid in some embodiments. The fatty alcohol is, for example, an aliphatic monohydric alcohol or an aliphatic dihydric alcohol, and is the aliphatic monohydric alcohol in some embodiments.

The aromatic carboxylic acid includes, for example, benzoic acid, toluic acid, cyclohexylbenzoic acid, phenylbenzoic acid, naphthoic acid, phthalic acid, or a mixture of them. One of them may be used alone, or two or more may be used in combination.

The aromatic carboxylic acid ester compound includes, for example, methyl benzoate, ethyl benzoate, propyl benzoate, methyl toluate, ethyl toluate, methyl cyclohexylbenzoate, ethyl cyclohexylbenzoate, methyl phenylbenzoate, ethyl phenylbenzoate, methyl naphthoate, ethyl naphthoate, dimethyl phthalate, diethyl phthalate, or a mixture of them. One of them may be used alone, or two or more may be used in combination.

A content of the carboxylic acid ester compound in the coolant composition is, for example, 10 mass % or more, and may be 30 mass % or more, 40 mass % or more, or 50 mass % or more. By setting the content of the carboxylic acid ester compound to 10 mass % or more, the insulation property and the heat transfer characteristic of the coolant composition can be improved. The content of the carboxylic acid ester compound in the coolant composition is, for example, 100 mass % or less, and may be 90 mass % or less.

The coolant composition according to the embodiment may include another nonaqueous base in addition to the carboxylic acid ester compound. The other nonaqueous base includes, for example, a mineral oil, a synthetic oil, or a mixture of them. The synthetic oil includes, for example, an ester synthetic oil, a synthetic hydrocarbon oil, a silicone oil, a fluorinated oil, or a mixture of them. One of them may be used alone, or two or more may be used in mixture.

The coolant composition according to the embodiment may include the mineral oil as the nonaqueous base in addition to the carboxylic acid ester compound. By including the mineral oil, the insulation property of the coolant composition can be improved. The mineral oil includes, for example, a paraffin mineral oil, a naphthenic mineral oil, or a mixture of them. One base oil may be used alone, or two or more base oils may be used in mixture.

While a kinematic viscosity (40° C.) of the mineral oil is not specifically limited, the kinematic viscosity is, for example, 0.5 to 100 mm²/s, and may be 0.5 to 20 mm²/s or 0.5 to 10 mm²/s.

A content of the mineral oil in the coolant composition may be 10 mass % or more, 20 mass % or more, 30 mass % or more, 40 mass % or more, or 50 mass % or more.

When the coolant composition includes the mineral oil, the content of the carboxylic acid ester compound in the coolant composition is 10 to 90 mass % and the content of the mineral oil in the coolant composition is 10 to 90 mass % in some embodiments. When the coolant composition includes the mineral oil, the content of the carboxylic acid ester compound in the coolant composition is 30 to 70 mass % and the content of the mineral oil in the coolant composition is 30 to 70 mass % in some embodiments. When the coolant composition includes the mineral oil, the content of the carboxylic acid ester compound in the coolant composition is 40 to 60 mass % and the content of the mineral oil in the coolant composition is 40 to 60 mass % in some embodiments.

The coolant composition according to the embodiment may include an optional component such, as an antioxidant agent, a rust inhibitor, a friction modifier, an anticorrosive, a viscosity index improver, a pour point depressant, a dispersing agent/surfactant, an antiwear agent, or a solid lubricant, in addition to the above-described components. A content of the optional component in the coolant composition is, for example, 0.1 to 20 mass %, and may be 10 mass % or less, 5 mass % or less, or 1 mass % or less.

A kinematic viscosity (20° C.) of the coolant composition according to the embodiment is, for example, 0.1 to 100 mm²/s, and may be 0.1 to 10 mm²/s.

Since the coolant composition is forcibly circulated in the cooling system, the viscosity may be lowered. The viscosity of the coolant composition can be adjusted by, for example, a viscosity and an amount of the mineral oil to be added. The kinematic viscosity (40° C.) of the coolant composition according to the embodiment may be 0.1 to 10 mm²/s.

A conductivity (20° C.) of the coolant composition according to the embodiment may be 0.1 μS/cm or less, 0.01 μS/cm or less, or 0.001 μS/cm or less.

### 2. Cooling System

The coolant composition according to the embodiment is used for the cooling system, and may be used for the cooling system mounted to a vehicle with traction motor. That is, an aspect of the embodiment is a cooling system where the coolant composition according to the embodiment is used as a refrigerant. An aspect of the embodiment is a cooling system for cooling heat generation equipment mounted to a vehicle with traction motor. An aspect of the embodiment is a vehicle with traction motor that includes the cooling system according to the embodiment and heat generation equipment cooled by the cooling system.

The “vehicle with traction motor” in this description includes both an electric vehicle and a hybrid vehicle. The electric vehicle includes only a traction motor as a power source without an engine. The hybrid vehicle includes both the traction motor and the engine as the power source. A fuel cell vehicle is also included in the “vehicle with traction motor.”

As one of the environmental measures, the vehicle with traction motor, such as the hybrid vehicle, the fuel cell vehicle, and the electric vehicle, that runs with driving force of the motor has attracted attention. In this vehicle, since the heat generation equipment, such as a motor, a generator, an inverter, a converter, and a battery, becomes to have a high temperature due to the heat generation, the heat generation equipment needs to be cooled. As described above, the coolant composition according to the embodiment is excellent in insulation property and heat transfer characteristic, and a secondary accident, such as a short circuit, is less likely to occur even when the coolant composition leaks due to an accident or the like. Therefore, the coolant composition according to the embodiment is usable for the cooling system of the vehicle with traction motor in some embodiments.

The cooling system includes, for example, a refrigerant pipe through which the coolant composition as a refrigerant flows, a reservoir tank that houses the coolant composition, a circulation device for circulating the coolant composition in a circulation passage, or a cooling device for decreasing the temperature of the coolant composition. The circulation device includes, for example, an electric pump. The cooling device includes, for example, a radiator, a chiller, or an oil cooler. A cooling object for the cooling device is the heat generation equipment, such as the inverter, the converter, the generator, the motor, and the battery.

The configuration of the cooling system is not specifically limited. The cooling system includes, for example, the refrigerant pipe, the reservoir tank, the electric pump, the radiator, and a cooling unit included in the heat generation equipment. The cooling unit is a unit to receive a heat from the heat generation equipment, and for example, the cooler 3 of FIG. 1 corresponds to the cooling unit. For example, after the coolant composition is pumped up from the reservoir tank by the electric pump, the heat generation equipment is cooled by the cooling unit, and subsequently, the coolant composition is returned to the reservoir tank via the radiator on a downstream side. Since the temperature of the coolant composition that has cooled the cooling unit increases, the temperature of the coolant composition that has increased in temperature is decreased by the radiator. A configuration where the oil cooler is disposed on the way of the refrigerant pipe to cool the motor by this oil cooler can be employed.

The cooling system according to the embodiment may be used for the vehicle with traction motor. That is, an aspect of the embodiment is a vehicle with traction motor that includes the cooling system according to the embodiment. An aspect of the embodiment is an electric vehicle, a hybrid vehicle, or a fuel cell vehicle that includes the cooling system according to the embodiment.

As described above, the coolant composition according to the embodiment is extremely excellent in insulation property, non-toxic, and less likely to cause corrosion. Thus, the coolant composition according to the embodiment is usable for the cooling system that has a cooling structure where the electronic equipment is at least partially (partially or completely) immersed in the coolant composition in some embodiments. The electronic equipment includes a power card, a CPU, and the like, which include semiconductor devices. Specific configurations of this cooling system can be found in U.S. Pat. No. 7,403,392 or US Patent Application Publication No. 2011-0132579 A. Specifically, an aspect of the embodiment is the vehicle with traction motor where the heat generation equipment includes the power card, and the coolant composition is in physical contact with the power card.

### Examples

While the following describes the embodiment with examples, the disclosure is not limited to the examples.

### <Material>

Ethyl octanoate (manufactured by Tokyo Chemical Industry)
Methyl decanoate (manufactured by Tokyo Chemical Industry)
Ethyl benzoate (manufactured by Tokyo Chemical Industry)
Mineral oil: kinematic viscosity (20° C.) 0.1 to 10 mm²/s
Conventional LLC (Toyota genuine, product name: Super Long-Life Coolant, including ethylene glycol and additive)
Ethylene glycol (manufactured by Tokyo Chemical Industry) (hereinafter also referred to as EG)
Ion exchanged water

### <Preparation Method>

Respective coolant compositions were prepared with compositions described in Tables 1-1 and 1-2 below.

### <Conductivity>

The conductivities of the respective coolant compositions at 20° C. were measured using a conductivity measuring machine (manufactured by Yokogawa Electric Corporation, SC72 Personal Handheld Conductivity Meter, sensor: SC72SN-11). Tables 1-1 and 1-2 indicate the results.

### <Heat Transfer Characteristic>

The heat transfer characteristics of the respective coolant compositions were compared by calculating the cooling performances of the radiator, the oil cooler, and the inverter, which used the respective coolant compositions as the refrigerants, with formulas below. Tables 1-1 and 1-2 indicate the results.

### (Cooling Performance in Radiator)

The cooling performances in the radiators using the respective coolant compositions as the refrigerants were calculated with the formula below. The refrigerants were adjusted to have inlet temperatures at 65° C. Other conditions were as follows. Ventilation volume to radiator: 4.5 m/sec, refrigerant flow rate: 10 L/min, temperature difference between refrigerant and external air: 40° C. (refrigerant: 65° C., external air: 25° C.).

$${Q}_{w} = \frac{{V}_{w} \cdot {γ}_{w} \cdot {10}^{- 3}}{60} \cdot {C}_{pw} \cdot ( {t}_{w 1} - {t}_{w 2} ) \qquad [ Math . 1 ]$$

Q_{W}: cooling performance, V_{W}: refrigerant flow rate, γ_{W}: refrigerant density, C_{PW}: refrigerant specific heat, t_{W1}: refrigerant inlet temperature, t_{W2}: refrigerant outlet temperature

### (Cooling Performance in Oil Cooler)

The cooling performances in the oil coolers using the respective coolant compositions as the refrigerants were calculated with the formula below. The refrigerants were adjusted to have the inlet temperatures at 30° C. Other conditions were as follows. Transmission oil flow rate: 6 L/min, refrigerant flow rate: 10 L/min, temperature difference between transmission oil and refrigerant: 30° C. (transmission oil: 60° C., refrigerant: 30° C.).

$${Q}_{w} = \frac{{V}_{w} \cdot {γ}_{w} \cdot {10}^{- 3}}{60} \cdot {C}_{pw} \cdot ( {t}_{w 1} - {t}_{w 2} ) \qquad [ Math . 2 ]$$

Q_{W}: cooling performance, V_{W}: refrigerant flow rate, γ_{W}: refrigerant density, C_{PW}: refrigerant specific heat, t_{W1}: refrigerant inlet temperature, t_{W2}: refrigerant outlet temperature

### (Cooling Performance in Inverter)

The cooling performances in the inverters using the respective coolant compositions as the refrigerants were calculated with the formula below. The refrigerants were adjusted to have the inlet temperatures at 65° C. Other conditions were as follows. Heat generation amount of inverter (power card): 500 W, refrigerant flow rate: 10 L/min.

$${Q}_{w} = \frac{{V}_{w} \cdot {γ}_{w} \cdot {10}^{- 3}}{60} \cdot {C}_{pw} \cdot ( {t}_{w 1} - {t}_{w 2} ) \qquad [ Math . 3 ]$$

Q_{W}: cooling performance, V_{W}: refrigerant flow rate, γ_{W}: refrigerant density, C_{PW}: refrigerant specific heat, t_{W1}: refrigerant inlet temperature, t_{W2}: refrigerant outlet temperature

TABLE 1-1

| | Component | Example 1 | Example 2 | Example 3 | Example 4 | Example 5 | Example 6 |
|---|---|---|---|---|---|---|---|
| Compositon | Ethyl Octanoate | 100 | — | — | 50 | — | — |
| (mass %) | Methyl Decanoate | — | 100 | — | — | 50 | — |
| | Ethyl Benzoate | — | — | 100 | — | — | 50 |
| | Conventional LLC | — | — | — | — | — | — |
| | (EG + additive) | | | | | | |
| | Ethylene Glycol | — | — | — | — | — | — |
| | Mineral Oil | — | — | — | 50 | 50 | 50 |
| | Ion Exchange Water | — | — | — | — | — | — |
| Sum | | 100 | 100 | 100 | 100 | 100 | 100 |
| Evaluation | Conductivity | <0.0009 | <0.0009 | <0.0009 | <0.0009 | <0.0009 | <0.0009 |
| | Cooling Performance (Radiator) | 228 | 211 | 211 | 208 | 200 | 203 |
| | Cooling Performance (Oil Converter) | 78 | 76 | 84 | 76 | 75 | 79 |
| | Cooling Performance (Inverter) | 5.8 | 5.8 | 6.2 | 5.6 | 5.6 | 5.8 |

TABLE 1-2

| | | | | | Comparative | Comparative | Comparative | Comparative |
|---|---|---|---|---|---|---|---|---|
| | Component | Example 7 | Example 8 | Example 9 | Example 1 | Example 2 | Example 3 | Example 4 |
| Composition | Ethyl Octanoate | 10 | — | — | — | — | — | — |
| (mass %) | Methyl Decanoate | — | 10 | — | — | — | — | — |
| | Ethyl Benzoate | — | — | 10 | — | — | — | — |
| | Conventional LLC | — | — | — | 50 | — | — | — |
| | (EG + Additive) | | | | | | | |
| | Ethylene Glycol | — | — | — | — | 50 | — | — |
| | Mineral Oil | 90 | 90 | 90 | — | — | 100 | — |
| | Ion Exchanged Water | — | — | — | 50 | 50 | — | 100 |
| Sum | | 100 | 100 | 100 | 100 | 100 | 100 | 100 |
| Evaluation | Conductivity | <0.0009 | <0.0009 | <0.0009 | 7000 | 0.6 | <0.0009 | 0.3 |
| | Cooling Performance (Radiator) | 199 | 199 | 198 | 371 | 368 | 197 | 458 |
| | Cooling Performance (Oil Cooler) | 74 | 74 | 74 | 115 | 114 | 73 | 124 |
| | Cooling Performance (Inverter) | 5.5 | 5.5 | 5.5 | 7.1 | 7.0 | 5.4 | 7.7 |

The coolant composition of any example had the conductivity less than 0.0009 μS/cm, and was extremely excellent in insulation property. Meanwhile, in comparative examples 1, 2 and 4 that had configurations of conventional coolant compositions (mixture of ethylene glycol and water, or water alone), the conductivities were high and the insulation properties were insufficient. The coolant composition of any example had the sufficient cooling performance required for a product. Especially, as the content of the carboxylic acid ester compound increased, the cooling performance was improved. Accordingly, it was proved that the coolant compositions according to the embodiment were excellent in insulation property and heat transfer characteristic.

Throughout the present specification, it should be understood that the singular forms also include the plural concept unless specifically stated otherwise. Thus, it should be understood that singular articles (for example, “a”, “an”, “the”, or the like in the case of English) also include the plural concept unless otherwise stated.

Upper limit values and/or lower limit values of respective numerical ranges described in this description can be appropriately combined to specify an intended range. For example, upper limit values and lower limit values of the numerical ranges can be appropriately combined to specify an intended range, upper limit values of the numerical ranges can be appropriately combined to specify an intended range, and lower limit values of the numerical ranges can be appropriately combined to specify an intended range.

While the embodiment has been described in detail, the specific configuration is not limited to the embodiment. Design changes within a scope not departing from the gist of the disclosure are included in the disclosure.

What is claimed is:

1. A coolant composition comprising
at least one carboxylic acid ester compound as a nonaqueous base,
wherein the coolant composition is substantially free of water.

2. The coolant composition according to claim 1,
wherein the carboxylic acid ester compound comprises at least one selected from the group consisting of an aliphatic carboxylic acid ester compound and an aromatic carboxylic acid ester compound, the aliphatic carboxylic acid ester compound is an ester of a saturated or unsaturated aliphatic carboxylic acid having 2 to 30 carbon atoms and a saturated or unsaturated fatty alcohol having 1 to 30 carbon atoms, and the aromatic carboxylic acid ester compound is an ester of an aromatic carboxylic acid having 6 to 20 carbon atoms and a saturated or unsaturated fatty alcohol having 1 to 30 carbon atoms.

3. The coolant composition according to claim 1,
wherein the carboxylic acid ester compound comprises at least one selected from an aliphatic carboxylic acid ester compound, the aliphatic carboxylic acid ester compound is an ester of a saturated or unsaturated aliphatic carboxylic acid having 2 to 30 carbon atoms and a saturated or unsaturated fatty alcohol having 1 to 30 carbon atoms.

4. The coolant composition according to claim 1,
wherein the carboxylic acid ester compound comprises at least one selected from an aromatic carboxylic acid ester compound, the aromatic carboxylic acid ester compound is an ester of an aromatic carboxylic acid having 6 to 20 carbon atoms and a saturated or unsaturated fatty alcohol having 1 to 30 carbon atoms.

5. The coolant composition according to claim 1,
wherein the saturated or unsaturated fatty alcohol comprises a monohydric alcohol.

6. The coolant composition according to claim 1,
wherein a content of the carboxylic acid ester compound in the coolant composition is 10 mass % or more.

7. The coolant composition according to claim 1, further comprising
a mineral oil.

8. The coolant composition according to claim 7,
wherein a content of the carboxylic acid ester compound in the coolant composition is 10 to 90 mass %, and
wherein a content of the mineral oil in the coolant composition is 10 to 90 mass %.

9. The coolant composition according to claim 7,
wherein a content of the carboxylic acid ester compound in the coolant composition is 30 to 70 mass %, and
wherein a content of the mineral oil in the coolant composition is 30 to 70 mass %.

10. The coolant composition according to claim 1,
wherein a conductivity at 20° C. is 0.1 μS/cm or less.

11. The coolant composition according to claim 1,
wherein a conductivity at 20° C. is 0.001 μS/cm or less.

12. A cooling system comprising the coolant composition according to claim 1 as a refrigerant.

13. The cooling system according to claim 12 for cooling heat generation equipment mounted to a vehicle with traction motor.

14. The cooling system according to claim 13,
wherein the heat generation equipment is an inverter, a converter, a generator, a motor, or a battery.

15. The cooling system according to claim 13,
wherein the heat generation equipment includes a power card, and the coolant composition is in physical contact with the power card.
